(19) United States
(12) Patent Application Publication
(10) Pub. No.: US 2023/0310106 A1
(43) Pub. Date: Oct. 5, 2023

(54) FORCEPS DEVICE AND BASE MEMBER

(71) Applicant: RIVERFIELD Inc., Tokyo (JP)

(72) Inventor: Kyohei TAKIKAWA, Tokyo (JP)

(73) Assignee: RIVERFIELD Inc., Tokyo (JP)

(21) Appl. No.: 18/330,710

(22) Filed: Jun. 7, 2023

Related U.S. Application Data
(63) Continuation of application No. PCT/JP2020/045719, filed on Dec. 8, 2020.

(51) Int. Cl. A61B 34/00; A61B 17/29

(52) U.S. Cl. CPC A61B 34/71; A61B 17/29; A61B 2017/2903; A61B 2017/2933; A61B 2017/2938

(57) ABSTRACT

A forceps device includes first and second grasping portions, a base member that holds the grasping portions, first and second wires that transmit driving forces to move the grasping portions, and a first guide pulley and a second guide pulley. The base member includes a partition part having holes including a first hole through which the first wire passes and a second hole that is closest to the first hole among the holes and through which the second wire passes, first and second arms extending from the partition part toward the grasping portions, respective ends of a support shaft being fixed to the first and second arms, and the first guide pulley being provided on the support shaft adjacent to the first arm and the second guide pulley being provided on the support shaft adjacent to the second arm and spaced apart from the first guide pulley.

### CROSS-REFERENCE TO RELATED APPLICATION

This Application is a continuation of International Application No. PCT/JP2020/045719, filed on Dec. 8, 2020 in the Japan Patent Office, the contents of which are incorporated by reference herein in its entirety.

### BACKGROUND

The present disclosure relates to a forceps device used for a manipulator of a surgical robot.

Medical treatments using robots (manipulators) have recently been proposed in order to reduce the burden on operators and save manpower in medical facilities. In the field of surgery, proposals have been made for surgical manipulator systems for operators to treat patients by operating remotely-controllable surgical manipulators.

Various surgical tools, such as a forceps for grasping human tissue during surgery, may be attached to leading ends of surgical manipulators.

### SUMMARY

It is an aspect to provide a novel technology for improving airtightness of a forceps device.

According to an aspect of one or more embodiments, there is provided a forceps device comprising a grasping part; a support that holds the grasping part; a rotating shaft that turnably supports the support; a base member that holds the rotating shaft; a plurality of grasping-portion wires that transmit driving forces to move the grasping part; a guide pulley that guides one or more of the plurality of grasping-portion wires; and a support shaft that rotatably supports the guide pulley. The base member includes a partition part having a plurality of grasping-portion-wire holes through which the respective grasping-portion wires pass; and a pair of arms extending from the partition part toward the grasping part, respective ends of the support shaft being fixed to the respective arms, wherein the plurality of grasping-portion wires include a first grasping-portion wire and a second grasping-portion wire, wherein the plurality of grasping-portion-wire holes include a first grasping-portion-wire hole through which the first grasping-portion wire passes, and a second grasping-portion-wire hole which is closest to the first grasping-portion-wire hole among the plurality of grasping-portion-wire holes and through which the second grasping-portion wire passes, and wherein the guide pulley guides the one or more of the plurality of grasping-portion wires so that the first grasping-portion wire is separated from the second grasping-portion wire.

According to an aspect of one or more embodiments, there is provided a base member arranged between a grasping part of a forceps device and a shaft of the forceps device, the base member comprising a partition part having a plurality of grasping-portion-wire holes through which a plurality of grasping-portion wires, respectively, pass, the plurality of grasping-portion wires transmitting driving forces to move the grasping part, wherein the plurality of grasping-portion-wire holes include a first grasping-portion-wire hole and a second grasping-portion-wire hole which is closest to the first grasping-portion-wire hole among the plurality of grasping-portion-wire holes, and when the partition part is viewed in a vertical direction, with respect to a center of the partition part, an angle between the first grasping-portion-wire hole and the second grasping-portion-wire hole is equal to or larger than 30°.

According to an aspect of one or more embodiments, there is provided a forceps device comprising a first grasping portion and a second grasping portion; a support that holds the first grasping portion and the second grasping portion; a first shaft that turnably supports the support; a base member that holds the first shaft; a first wire and a second wire that transmit driving forces to move the first grasping portion and the second grasping portion; a first guide pulley and a second guide pulley; and a support shaft that rotatably supports the first guide pulley and the second guide pulley. The base member includes a partition part having a plurality of holes including a first hole through which the first wire passes and a second hole that is closest to the first hole among the plurality of holes and through which the second wire passes; and a first arm and a second arm extending from the partition part toward the first grasping portion and the second grasping portion, respective ends of the support shaft being fixed to the first arm and the second arm, and the first guide pulley being provided adjacent to the first arm and the second guide pulley being provided adjacent to the second arm and spaced apart from the first guide pulley

### BRIEF DESCRIPTION OF DRAWINGS

FIG. 1 is a perspective view of a forceps device according to some embodiments.

FIG. 2 is a front view of the forceps device of FIG. 1 as viewed in a direction A.

FIG. 3 is a side view of the forceps device of FIG. 1 as viewed in a direction B.

FIG. 4 is a side view of the forceps device of FIG. 1 as viewed in a direction C.

FIG. 5 is a cross-sectional view of the forceps device illustrated in FIG. 3 along D-D.

FIG. 6 is an enlarged view of a region R in FIG. 5.

FIG. 7 is a front view of a transmission pulley according to some embodiments.

FIG. 8 is a cross-sectional view of the transmission pulley illustrated in FIG. 7 along E-E.

FIG. 9 is a perspective view of a support according to some embodiments.

FIG. 10 is a flowchart explaining a method for manufacturing the forceps device according to some embodiments.

FIG. 11 is a perspective view illustrating a state in which a pair of jaw parts facing each other are to be fitted according to some embodiments.

FIG. 12 is a perspective view of the forceps device illustrated in FIG. 1 as viewed in a direction H1.

FIG. 13 is a perspective view of the forceps device illustrated in FIG. 1 as viewed in a direction H2.

FIG. 14 is a perspective view of the forceps device illustrated in FIG. 1 as viewed in a direction H3.

FIG. 15 is a perspective view of the forceps device illustrated in FIG. 1 as viewed in a direction opposite the direction H2.

FIG. 16 is a schematic view of the forceps device illustrated in FIG. 3 omitting a base member.

FIG. 17 is a schematic view of the forceps device illustrated in FIG. 4 omitting the base member.

FIG. 18 is a schematic view of the forceps device illustrated in FIG. 1 omitting wires.

FIG. 19 is a perspective view of the base member according to some embodiments.

FIG. 20 is a top view of the base member illustrated in FIG. 19 as viewed in a direction J1.

FIG. 21 is a bottom view of the base member illustrated in FIG. 19 as viewed in a direction J2.

FIG. 22 is a perspective view of a jig to be used for producing a sealing member.

FIG. 23 is a perspective view illustrating a state in which the jig illustrated in FIG. 22 is attached to the base member.

FIG. 24 is a schematic diagram illustrating injection of sealing resin in a state in which the jig is attached to the base member.

### DETAILED DESCRIPTION

As discussed above, surgical tools may include a forceps for grasping human tissue during surgery. For example, a forceps may include two jaw parts serving as grasping portions, two disks having different outer diameters from each other provided on the respective jaw parts, and wires running over the disks. The wires may be pulled to open or close the jaw parts.

When an operation is performed with the forceps inserted into a patient's abdomen, an air pressure inside the abdominal cavity of the patient may be increased to be higher than an atmospheric pressure in order to secure the surgical field. If, however, gas inside the abdominal cavity leaks out of the patient's body through a leading end of the forceps and a shaft, the air pressure inside the abdominal cavity cannot be increased to be higher than the atmospheric pressure. In particular, in a case where the jaw parts at the leading end of the forceps are opened and closed by forward and backward movements of wires running through the shaft, it is advantageous to provide sealing based on a consideration of the wire movements.

It is an aspect to provide a novel technology for improving the airtightness of the grasping part side of a forceps device.

According to some embodiments, a forceps device may include a grasping part; a support that holds the grasping part; a first rotating shaft that turnably supports the support; a base member that holds the first rotating shaft; a plurality of grasping-portion wires for transmitting driving forces to move the grasping part; a guide pulley for guiding one or some of the plurality of grasping-portion wires; and a support shaft for rotatably supporting the guide pulley. The base member may include a partition part having a plurality of grasping-portion-wire holes through which the respective grasping-portion wires pass; and a pair of arms extending from the partition part toward the grasping part, respective ends of the support shaft being fixed to the respective arms. The plurality of grasping-portion wires may include a first grasping-portion wire and a second grasping-portion wire. The plurality of grasping-portion-wire holes may include a first grasping-portion-wire hole through which the first grasping-portion wire passes, and a second grasping-portion-wire hole which is closest to the first grasping-portion-wire hole and through which the second grasping-portion wire passes. The guide pulley may guide the one or some of the plurality of grasping-portion wires so that the first grasping-portion wire is separated from the second grasping-portion wire.

Accordingly, the first grasping-portion-wire hole can be separated from the second grasping-portion-wire hole. This separation allows a sealing member having holes to be molded with high accuracy on one side of the partition part having the holes and increases the airtightness on the grasping part side, for example.

According to some embodiments, when the partition part is viewed in a vertical direction, an angle at a center of the partition part between the first grasping-portion-wire hole and the second grasping-portion-wire hole may be equal to or larger than 30°. As a result, the distance between the first grasping-portion-wire hole and the second grasping-portion-wire hole becomes large, which reduces such defects as integration of holes when the sealing member with a plurality of holes is molded, for example.

According to some embodiments, the base member may include a sealing member on one side with respect to the partition part, and the sealing member may have a plurality of sealing holes at positions corresponding to the plurality of grasping-portion-wire holes. This configuration enables sealing with the sealing member with the grasping-portion wires passing through the base member.

According to some embodiments, the sealing holes may each have a diameter smaller than a diameter of a corresponding one of the grasping-portion-wire holes and smaller than a diameter of a corresponding one of the grasping-portion wires. As a result, even when a gap is present between a grasping-portion-wire hole and a grasping-portion wire, a sealing hole and the grasping-portion wire are in close contact with each other, which reduces leakage of gas from the grasping part side to the outside of the forceps device via the base member.

According to some embodiments, the sealing member may be a silicone resin film.

According to some embodiments, a pair of support wires for transmitting driving forces to turn the support may further be included, and the partition part may have a pair of support-wire holes through which the pair of support wires, respectively, pass, and when the partition part is viewed in a vertical direction, an angle at a center of the partition part between each of the support-wire holes and the grasping-portion-wire hole that is closest to the support-wire hole may be equal to or larger than 30°. As a result, the support-wire holes and the grasping-portion-wire holes can be separated from each other. Thus, the sealing member having holes may be molded with high accuracy on one side with respect to the partition part having holes, and the airtightness on the grasping part side is increased, for example.

According to some embodiments, a base member may be arranged between a grasping part and a shaft of a forceps device. The base member may include a partition part having a plurality of grasping-portion-wire holes through which a plurality of grasping-portion wires, respectively, pass, the grasping-portion wires transmitting driving forces to move the grasping part. The plurality of grasping-portion-wire holes may include a first grasping-portion-wire hole and a second grasping-portion-wire hole which is closest to the first grasping-portion-wire hole. When the partition part is viewed in a vertical direction, an angle at a center of the partition part between the first grasping-portion-wire hole and the second grasping-portion-wire hole is equal to or larger than 30°.

Accordingly, the first grasping-portion-wire hole and the second grasping-portion-wire hole can be widely separated from each other. This separation allows a sealing member having holes to be molded with high accuracy on one side of the partition part having the holes and increases the airtightness on the grasping part side, for example. In addition, such defects as integration of holes when the sealing member with a plurality of holes is molded, for example, can be reduced.

According to some embodiments, the partition part may have a pair of support-wire holes through which a pair of support wires, respectively, pass, the support wires transmitting driving forces for turning a support that holds the grasping part, and when the partition part is viewed in the vertical direction, an angle at the center of the partition part between each of the support-wire holes and the grasping-portion-wire hole that is closest to the support-wire hole may be equal to or larger than 30°. As a result, the support-wire holes and the grasping-portion-wire holes can be separated from each other. Thus, the sealing member having holes is molded with high accuracy on one side with respect to the partition part having holes, and the airtightness on the grasping part side is increased, for example.

According to some embodiments, a pair of arms extending from the partition part toward the grasping part may further be included. The arms may each have a fitting hole, respective ends of a support shaft that rotatably supports a pair of guide pulleys being fitted into the fitting holes. As a result, the pair of arms are fixed via the support shaft, which increases the stiffness of the base member.

According to some embodiments, a method, a device, a system and the like consistent with the above configurations may also be provided.

According to the various embodiments described herein, a forceps device with high airtightness on the grasping part side can be achieved.

Various embodiments will now be described with reference to the drawings. Components, members, and processes that are the same as or equivalent to each other illustrated in the drawings are represented by the same reference numerals, and redundant explanation will not be repeated where appropriate for conciseness. The present disclosure is not to limit the various embodiments described herein, but rather the various embodiments are provided as examples, and any individual feature or any combination of features described in the various embodiments is not necessarily essential.

### Forceps Device

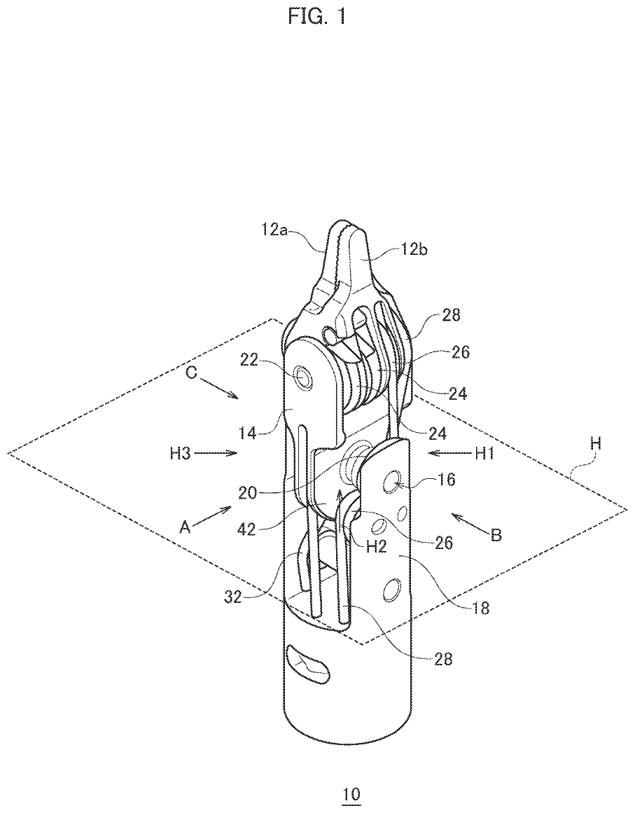

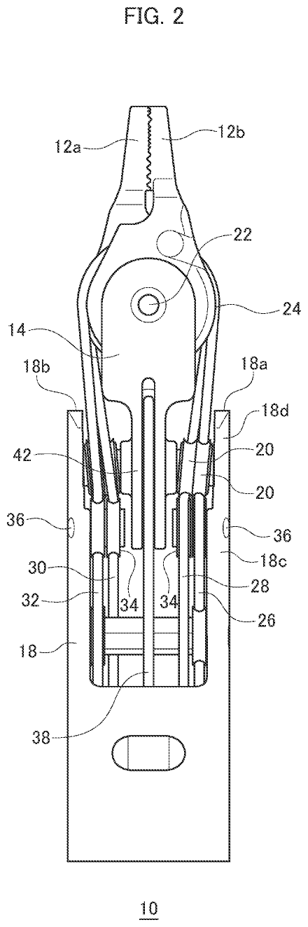

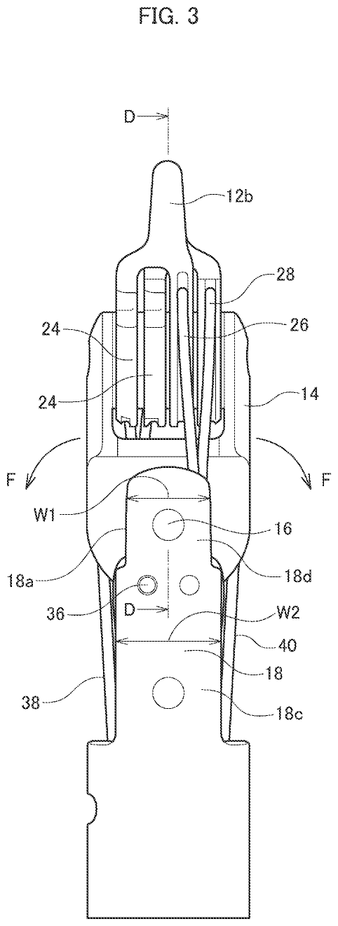

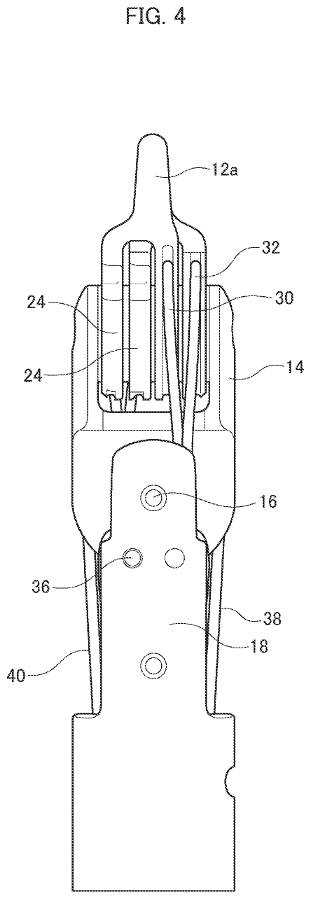

FIG. 1 is a perspective view of a forceps device according to some embodiments. FIG. 2 is a front view of the forceps device of FIG. 1 as viewed in a direction A. FIG. 3 is a side view of the forceps device of FIG. 1 as viewed in a direction B. FIG. 4 is a side view of the forceps device of FIG. 1 as viewed in a direction C.

The forceps device 10 illustrated in the drawings includes a pair of grasping portions 12a and 12b, a support 14 that holds the pair of grasping portions 12a and 12b, a first rotating shaft 16 that turnably supports the support 14, a base member 18 that holds the first rotating shaft 16, four transmission pulleys 20 arranged coaxially with the first rotating shaft 16, a second rotating shaft 22 that turnably supports the pair of grasping portions 12a and 12b and is held by the support 14, four jaw pulleys 24 supported coaxially with the second rotating shaft 22, four wires 26, 28, 30 and 32 running over the four transmission pulleys 20 and the four jaw pulleys 24, and wires 38 and 40 for rotating the support 14 about the first rotating shaft 16.

### Transmission Pulleys 20

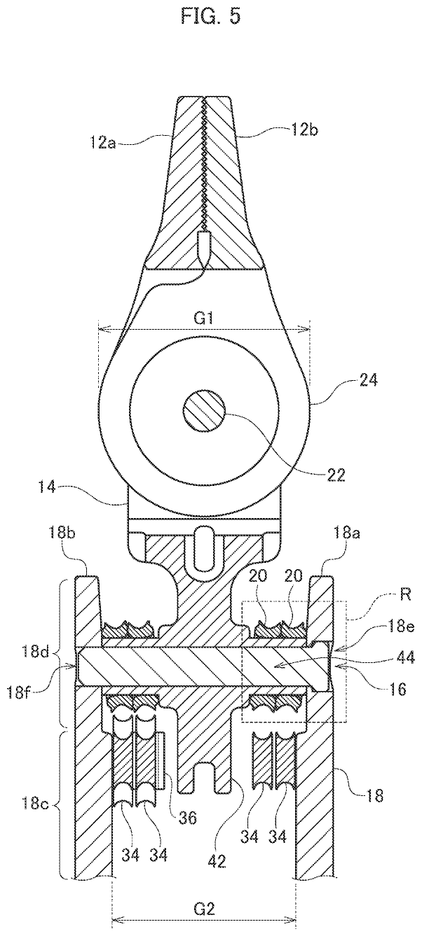

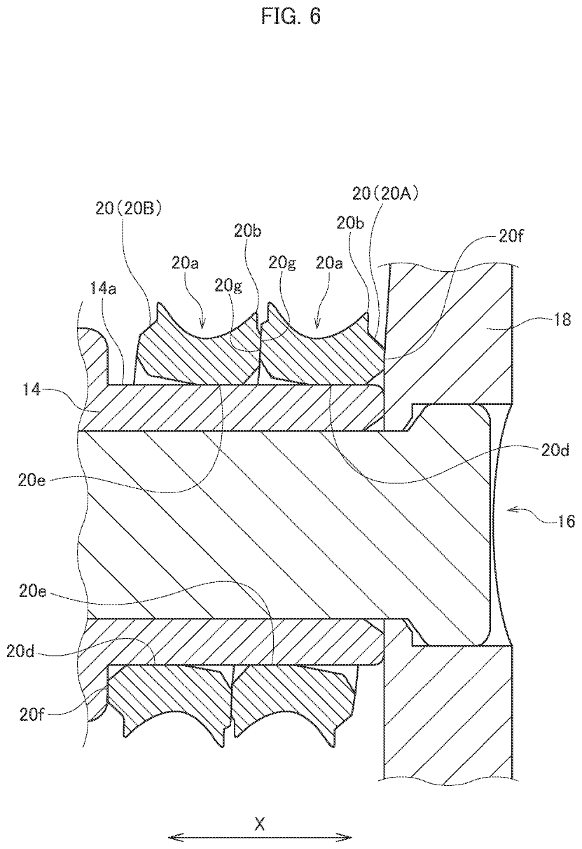

FIG. 5 is a cross-sectional view of the forceps device 10 illustrated in FIG. 3 along D-D. FIG. 6 is an enlarged view of a region R in FIG. 5. As illustrated in FIGS. 5 and 6, the transmission pulleys 20 are rotatably supported by the first rotating shaft 16 with an annular part 14a having a cylindrical shape, which is part of the support 14, therebetween and at an angle with respect to the first rotating shaft 16. Thus, the respective wires 26, 28, 30 and 32 have small substantial fleet angles with respect to the transmission pulleys 20, and therefore become less likely to interfere with edges 20b of guide grooves 20a of the transmission pulleys 20. In some embodiments, the transmission pulleys 20 may be directly supported by the first rotating shaft 16 without the support 14 therebetween.

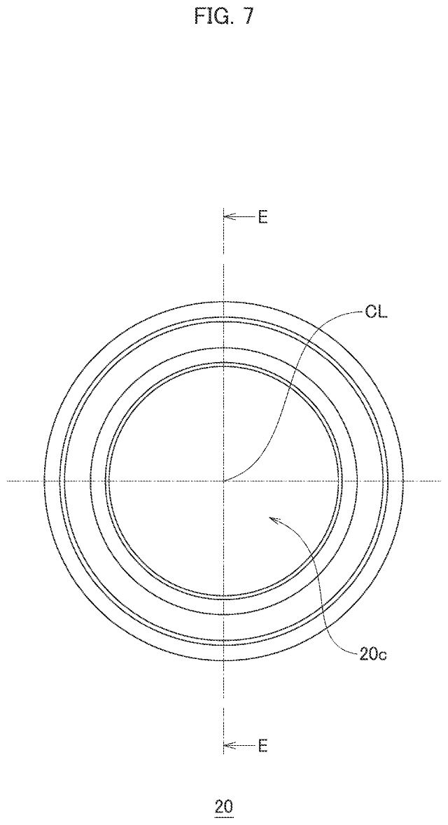

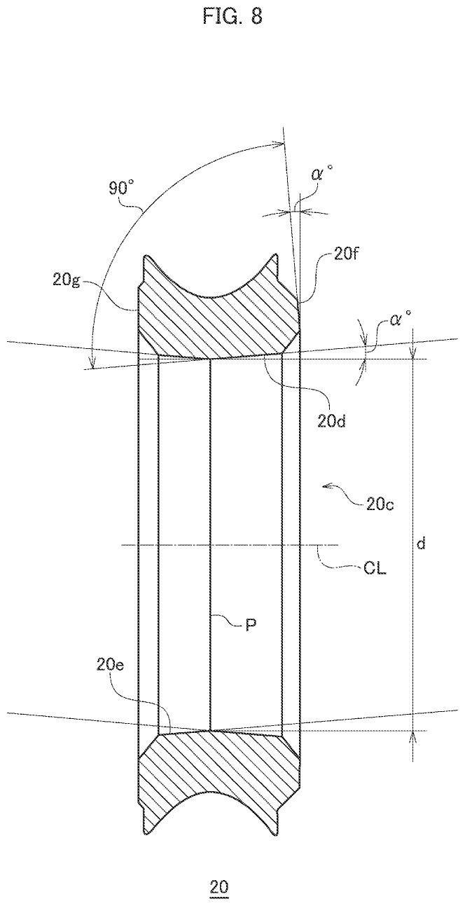

FIG. 7 is a front view of a transmission pulley 20 according to some embodiments. FIG. 8 is a cross-sectional view of the transmission pulley 20 illustrated in FIG. 7 along E-E. Each transmission pulley 20 according to some embodiments has an inner circumferential face 20d tilted with respect to a center line CL of a circular opening 20c thereof through which the first rotating shaft 16 extends. The inner circumferential face 20d slides relative to the annular part 14a of the support 14 into which the first rotating shaft 16 is inserted. Thus, the transmission pulleys 20 are autonomously tilted due to the forces received from wires having fleet angles. In a case where the transmission pulleys 20 are supported directly by the first rotating shaft 16, the inner circumferential face 20d of each transmission pulley 20 slides directly relative to the outer circumferential face of the first rotating shaft 16.

As illustrated in FIG. 8, the inner circumferential face 20d of each transmission pulley 20 is formed so that the inner diameter d of the transmission pulley 20 gradually increases from the position of a plane P, which is perpendicular to the center line CL and at which the inner diameter d is smallest, as the position is farther along the center line CL from the plane P. In other words, the inner circumferential face 20d may have a conical shape, an inverted cone shape, or a tapered shape. In some embodiments, an inner circumferential face 20e opposite the inner circumferential face 20d with respect to the plane P is also processed to have a shape similar to the inner circumferential face 20d.

Note that each of the transmission pulleys 20 according to some embodiments is formed such that the angle α between the inner circumferential face 20d or the inner circumferential face 20e and the center line CL is within a range from 3 to 7 degrees. In some embodiments, one end face 20f of each transmission pulley 20 is tilted at the angle α with respect to the plane P, and the angle between the end face 20f and the inner circumferential face 20d is 90°. Furthermore, the other end face 20g of the transmission pulley 20 is parallel to the plane P (perpendicular to the center line CL). Each transmission pulley 20 is therefore an annular member that is asymmetric with respect to the plane P.

As illustrated in FIGS. 5 and 6, the transmission pulleys 20 according to some embodiments are arranged in such a manner that two transmission pulleys 20 are arranged adjacent to each other on each side of the support 14 with the support 14 therebetween. In some embodiments, as illustrated in FIG. 6, the end faces 20g of two transmission pulleys 20 that are arranged adjacent to each other on the annular part 14a face each other. As a result, one (a transmission pulley 20B) of two transmission pulleys 20 that are adjacent to each other is in contact with and tilted with respect to the support 14, and the other (a transmission pulley 20A) of the two transmission pulleys 20 adjacent to each other is in contact with and tilted with respect to the base member 18. Thus, when a transmission pulley 20 is tilted, the transmission pulley 20 is accurately positioned in a first rotating shaft direction X.

As illustrated in FIG. 2, the transmission pulleys 20 are each tilted with respect to the first rotating shaft 16 so that the wires 26 and 28 running over the transmission pulleys 20 extend out toward the outer circumference of the jaw pulleys 24. As a result, even in a case where jaw pulleys 24 having a large diameter that are likely to increase the operation torque of the pair of grasping portions 12a and 12b are used, interference of wires at the transmission pulleys 20 can be reduced.

Next, the base member 18 will be described. As illustrated in FIG. 2 and FIG. 5, the base member 18 according to some embodiments includes a pair of arms 18a and 18b that hold respective ends of the first rotating shaft 16, and third rotating shafts 36 that are held by the pair of arms 18a and 18b, respectively, and rotatably support four upstream pulleys 34 located upstream of the transmission pulleys 20. Note that a third rotating shaft 36 according to some embodiments is provided on each of the pair of arms 18a and 18b.

The arms 18a and 18b each have a base part 18c holding the third rotating shaft 36, and a distal end part 18d that holds the first rotating shaft 16 and that is thinner than the base part 18c. In other words, the distance between the distal end parts 18d is larger than the distance between the base parts 18c. Thus, as illustrated in FIG. 3, even when the wires 26 and 28 having the fleet angles are bent together with the support 14 around the first rotating shaft 16 (in the direction of an arrow F in FIG. 3), the wires 26 and 28 are less likely to interfere with the arms 18a and 18b.

Furthermore, the circumferential width W1 of the distal end part 18d of the arm 18a (the arm 18b) is smaller than the circumferential width W2 of the base part 18c thereof. Thus, a U-shaped recess is formed at the distal end part 18d at which interference with a wire needs to be addressed, and the circumferential width of the distal end part 18d is made larger than the circumferential width of the base part 18c, which minimizes deterioration of the stiffness of the arm.

In some embodiments, as illustrated in FIG. 5, the jaw pulleys 24 according to some embodiments have an outer diameter G1, which is larger than the distance between the base parts 18c of the pair of arms 18a and 18b. Thus, in the forceps device 10 according to some embodiments, the jaw pulleys 24 having a large outer diameter relative to the inner diameter of the cylindrical base member 18 may be used.

### Easiness of Assembly of Support

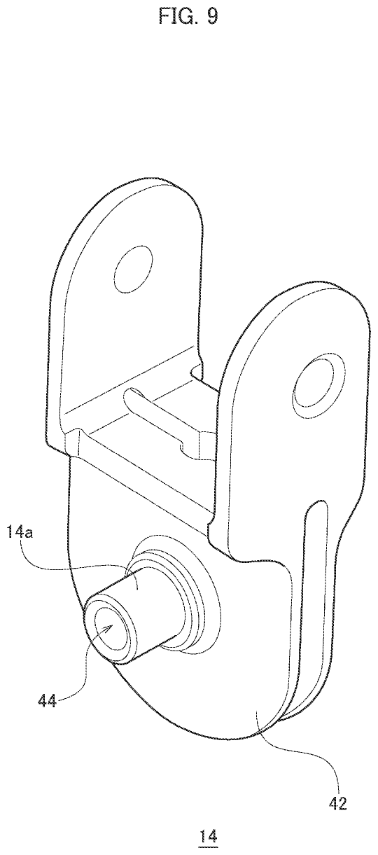

FIG. 9 is a perspective view of the support according to some embodiments. As illustrated in FIGS. 5 and 6, the forceps device 10 according to some embodiments includes the pair of grasping portions 12a and 12b, the support 14 that holds the pair of grasping portions 12a and 12b, the first rotating shaft 16 that turnably supports the support 14, the base member 18 that holds the first rotating shaft 16, and a plurality of transmission pulleys 20 arranged coaxially with the first rotating shaft 16. As illustrated in FIG. 9, the support 14 has the annular part 14a, which is a cylindrical part through which the first rotating shaft 16 extends. The transmission pulleys 20 are rotatably supported by the outer circumference of the annular part 14a. Note that each rotating shaft is not limited to a shaft that rotates by itself, but may be any shaft that is the center of rotation of a member supported thereby, and may be a shaft fixed to another member.

In the forceps device 10 having such a structure, the support 14 in a state in which a plurality of transmission pulleys 20 are supported by the outer circumference of the annular part 14a is held by the base member 18 with the first rotating shaft 16 therebetween. This configuration facilitates improvement in the easiness of assembly as compared with a case where the support 14 is held directly by the base member 18.

In some embodiments, the base member 18 has the pair of arms 18a and 18b facing each other. The first rotating shaft 16 is firmly fixed in such a manner that the axial ends thereof are press-fitted to the pair of arms 18a and 18b. As a result, because the distal ends of the pair of arms 18a and 18b, which can be free ends, are fixed by the rotating shaft, the stiffness of the whole base member 18 increases.

Each of the wires 26, 28, 30 and 32 transmits a driving force to the grasping portion 12a or the grasping portion 12b to move the grasping portion 12a or the grasping portion 12b. Specifically, the wire 26 and the wire 32 run over the jaw pulleys 24 for the grasping portion 12b, and the grasping portion 12b moves in an opening direction when the wire 26 is pulled and moves in a closing direction when the wire 32 is pulled. The wire 28 and the wire 30 run over the jaw pulleys 24 for the grasping portion 12a, and the grasping portion 12a moves in a closing direction when the wire 28 is pulled and moves in an opening direction when the wire 30 is pulled.

In some embodiments, each of the four transmission pulleys 20 has a corresponding one of the wires 26, 28, 30 and 32 placed thereover. Each of the wires 26, 28, 30 and 32 runs over the corresponding one of the transmission pulleys 20 so that the normal force applied to the support 14 from the first rotating shaft 16 is reduced when the grasping portions 12a and 12b are moved. More specifically, as illustrated in FIG. 2, each of the wires 26, 28, 30 and 32 passes between the corresponding ones of the transmission pulleys 20 and upstream pulleys 34 and runs over the corresponding one of the transmission pulleys 20 from a lower side thereof (a side opposite the grasping portion 12a or 12b with respect to the first rotating shaft 16).

Thus, when tension is applied to at least any one of the wires 26, 28, 30 and 32 for moving the grasping portions 12a and 12b, the tension causes a force acting on the support 14 via the transmission pulleys 20. In the case of the forceps device 10 according to some embodiments, the tension applied to the wires 26, 28, 30 and 32 causes a force acting on the support 14 in directions toward the grasping portions 12a and 12b.

The support 14 is formed integrally with a support pulley 42 over which the wires 38 and 40 for transmitting a driving force for rotating the support about the first rotating shaft 16 run. Thus, when one of the wires 38 and 40 is pulled to turn the support 14, the tension of the wire 38 or 40 running over the support pulley 42 presses the support 14 toward the first rotating shaft 16. A normal force received by the support 14 from the first rotating shaft 16 is thus generated, which contributes to an increase in frictional force.

In the forceps device 10 according to some embodiments, however, the tension of the wires 26, 28, 30 and 32 for moving the grasping portions 12a and 12b causes a force acting on the transmission pulleys 20 in the upward direction in FIG. 2 as described above. The normal force caused by the wires 38 and 40 is reduced by the force acting on the transmission pulleys 20, which reduces the frictional force when the support 14 turns about the first rotating shaft 16. Consequently, the movement of the grasping portions 12a and 12b becomes smoother, and the controllability of the forceps device 10 improves.

### Method for Producing Forceps Device

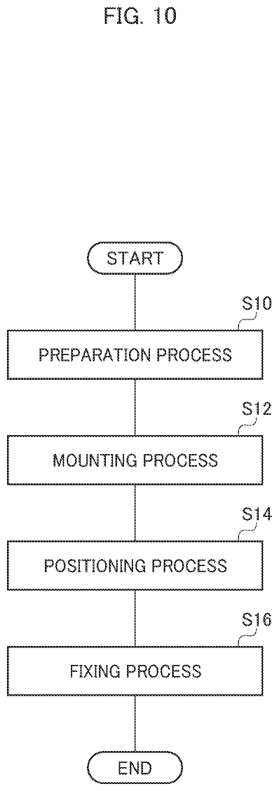

Next, a method for producing the forceps device 10 according to some embodiments will be explained. FIG. 10 is a flowchart explaining a method for manufacturing the forceps device according to some embodiments. In the method, the support 14 holding the grasping portions 12a and 12b is first prepared (S10). Subsequently, a plurality of transmission pulleys 20 are mounted on the outer circumference of the annular part 14a through which a through-hole 44 (see FIG. 5) is formed (S12). The first rotating shaft 16 that turnably supports the support 14 is to be inserted in the through-hole 44. Subsequently, the support 14 is positioned so that holes 18e and 18f into which the first rotating shaft 16 is to be inserted and the through-hole 44 are linearly aligned with respect the base member 18 through which the holes 18e and 18f are formed (S14). The first rotating shaft 16 is then inserted into the hole 18e, and the respective ends of the first rotating shaft 16 are ultimately press-fitted into the holes 18e and 18f of the base member 18, so that the first rotating shaft 16 is firmly fixed to the base member 18 (S16). Note that a process of passing wires over the pulleys may be performed at a timing that does not interfere with the manufacture described above.

According to the manufacturing method, such a process as sandwiching a support by base member parts into which a base member is divided and then bonding the base member parts to each other need not be performed. In some embodiments, such a process as coaxially arranging holes of a support, pulleys and a base member, and press-fitting a rotating shaft into the support while keeping the positions of the holes, which involves difficult adjustment, need not be performed.

### Grasping Portions

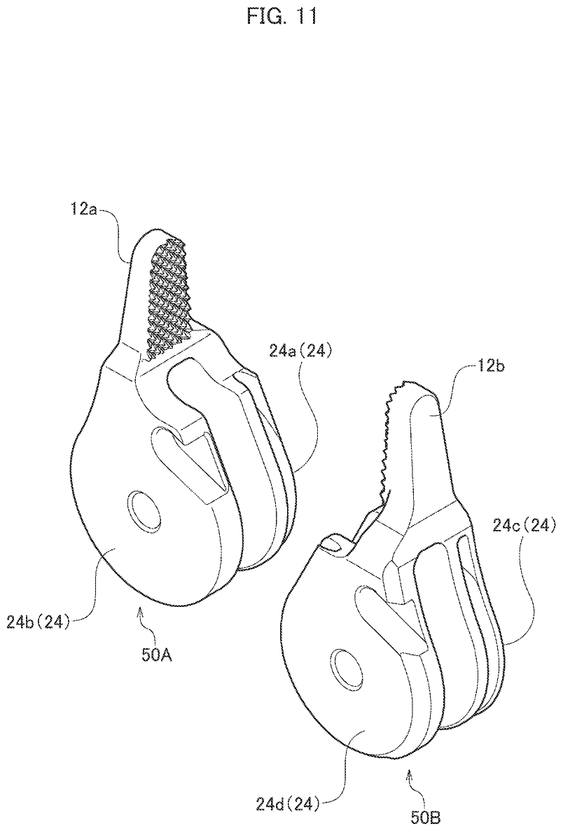

Next, jaw parts constituting the grasping portions according to some embodiments will be described in detail. FIG. 11 is a perspective view illustrating a state in which a pair of jaw parts facing each other are to be fitted according to some embodiments. Jaw parts 50A and 50B illustrated in FIG. 11 are parts having substantially the same shapes as each other. The jaw parts 50A and 50B have the grasping portion 12a and the grasping portion 12b, respectively, which move relative to each other to grasp an object. The grasping portion 12a is continuous with a jaw pulley 24a (first grasping portion pulley) and a jaw pulley 24b (second grasping portion pulley), which are formed in a bifurcated shape, and the grasping portion 12a and the jaw pulleys 24a and 24b integrally constitute the jaw part 50A. In some embodiments, the grasping portion 12b is continuous with a jaw pulley 24c (third grasping portion pulley) and a jaw pulley 24d (fourth grasping portion pulley), which are formed in a bifurcated shape, and the grasping portion 12b and the jaw pulleys 24c and 24d integrally constitute the jaw part 50B.

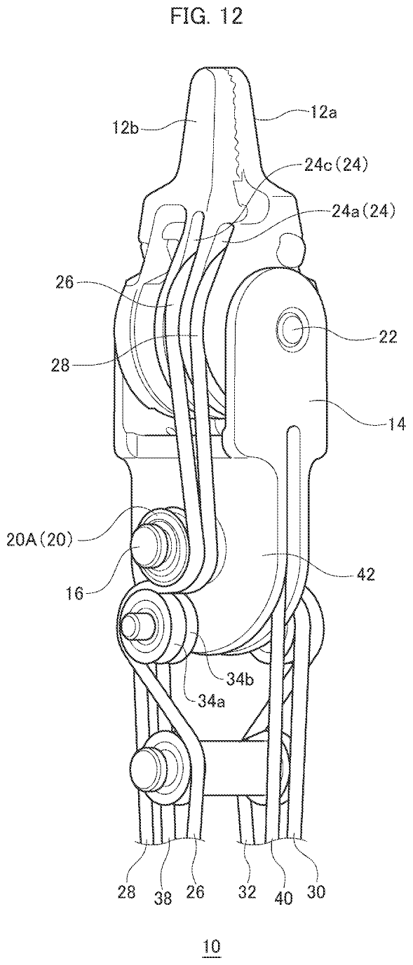

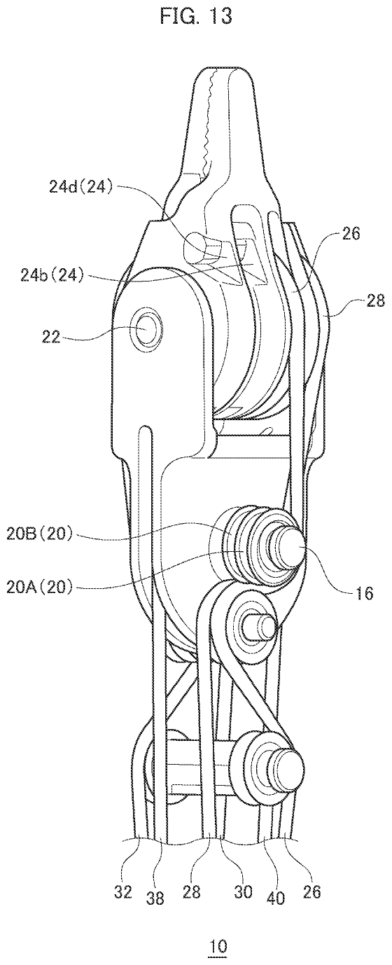

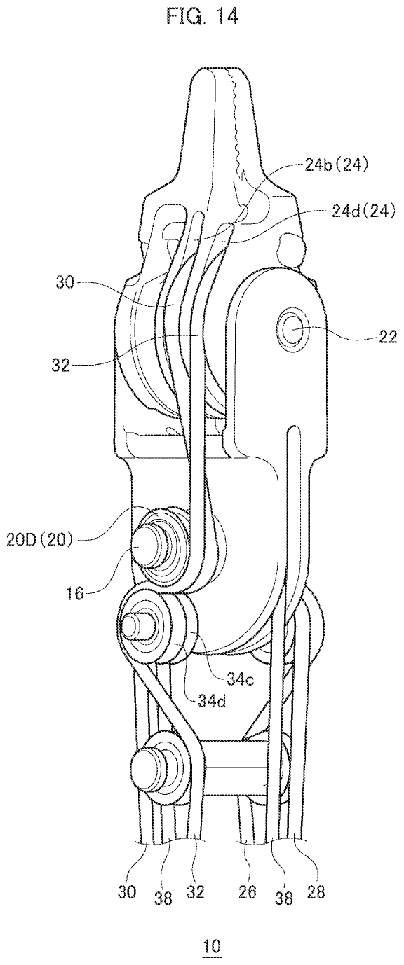

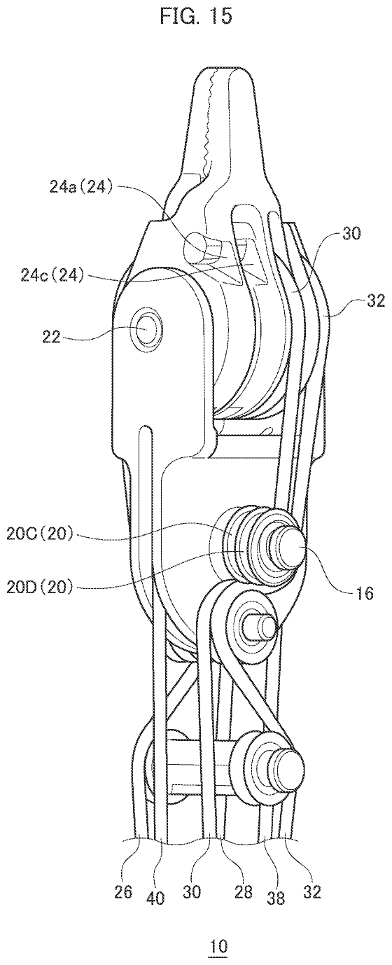

FIG. 12 is a perspective view of the forceps device 10 illustrated in FIG. 1 as viewed in a direction H1. FIG. 13 is a perspective view of the forceps device 10 illustrated in FIG. 1 as viewed in a direction H2. FIG. 14 is a perspective view of the forceps device 10 illustrated in FIG. 1 as viewed in a direction H3. FIG. 15 is a perspective view of the forceps device 10 illustrated in FIG. 1 as viewed in a direction opposite the direction H2. Note that the directions H1 to H3 are directions within a horizontal plane H including the first rotating shaft 16. In some embodiments, the base member 18 is not illustrated in FIGS. 12 to 15.

As illustrated in FIGS. 12 to 15, the jaw pulley 24a, the jaw pulley 24c, the jaw pulley 24b, and the jaw pulley 24d are rotatably supported in this order by the second rotating shaft 22. In some embodiments, the transmission pulley 20A, the transmission pulley 20B, the transmission pulley 20C, and the transmission pulley 20D are rotatably supported in this order by the first rotating shaft 16. The wires 26, 28, 30 and 32, which are grasping-portion wires for transmitting driving forces for causing the grasping portions 12a and 12b to perform opening and closing movements, run between the transmission pulleys 20A to 20D and the grasping portions 12a and 12b without other pulleys. Note that the driving forces are input from an actuator unit outside of the forceps device 10. This configuration can shorten the distance between the first rotating shaft 16 and the second rotating shaft 22 as illustrated in FIG. 12, etc., and can therefore increase the torque (operation force) of the grasping portions 12a and 12b. Consequently, the forceps device 10 with high controllability of the grasping portions 12a and 12b can be provided.

### How Wires Run Over Pulleys

Next, the manner in which the wires 26, 28, 30 and 32 according to some embodiments run over the pulleys will be described in detail. The wire 26 runs between the transmission pulley 20A and the jaw pulley 24c. The wire 28 runs between the transmission pulley 20B and the jaw pulley 24a. The wire 30 runs between the transmission pulley 20C and the jaw pulley 24b. The wire 32 runs between the transmission pulley 20D and the jaw pulley 24d.

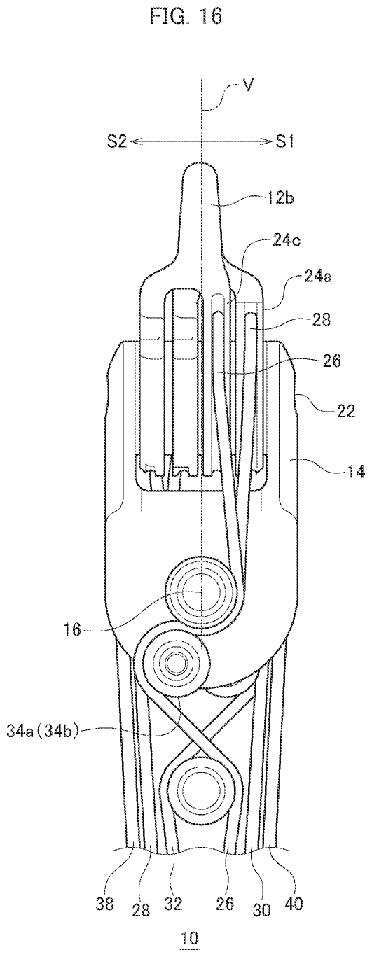

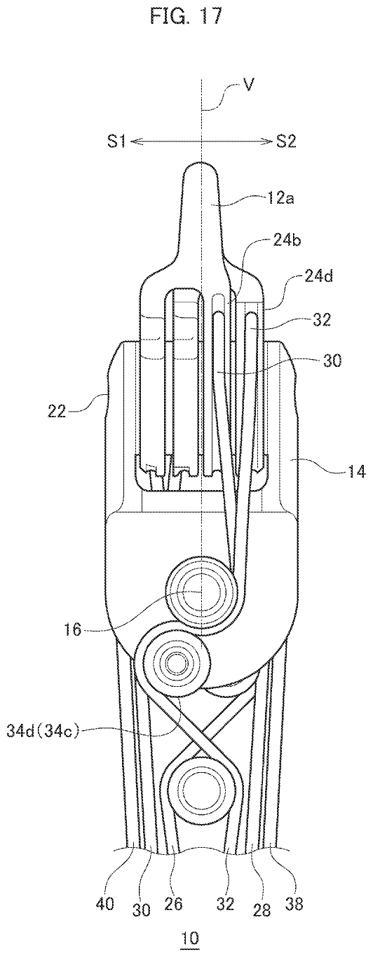

FIG. 16 is a view of the forceps device 10 illustrated in FIG. 3 without illustration of the base member 18. FIG. 17 is a view of the forceps device 10 illustrated in FIG. 4 without illustration of the base member.

As illustrated in FIGS. 12, 13, and 16, the wire 26 is passed over a first side 51 of the transmission pulley 20A with respect to a vertical cross section V including the first rotating shaft 16 and being perpendicular to the second rotating shaft 22, and then fixed to the jaw pulley 24c located on the first side 51 with respect to the vertical cross section V. The wire 28 is passed over the first side 51 of the transmission pulley 20B with respect to the vertical cross section V, and then fixed to the jaw pulley 24a located on the first side 51 with respect to the vertical cross section V.

In some embodiments, as illustrated in FIGS. 14 to 16, the wire 30 is passed over the second side S2 of the transmission pulley 20C with respect to the vertical cross section V, and then fixed to the jaw pulley 24b located on the second side with respect to the vertical cross section V. The wire 32 is passed over the second side S2 of the transmission pulley 20D with respect to the vertical cross section V, and then fixed to the jaw pulley 24d located on the second side S2 with respect to the vertical cross section V. As a result, the four grasping-portion wires are fixed to the four grasping portion pulleys, respectively, without intersecting with each other. In some embodiments, the four grasping-portion wires running over the transmission pulleys 20A to 20D are fixed to the four grasping portion pulleys, respectively, with small fleet angles.

Furthermore, the forceps device 10 includes the upstream pulley 34a on the upstream side of the transmission pulley 20A (on the side opposite the grasping portions) and on the second side S2 with respect to the vertical cross section V, the upstream pulley 34b on the upstream side of the transmission pulley 20B and on the second side S2 with respect to the vertical cross section V, the upstream pulley 34c on the upstream side of the transmission pulley 20C and on the first side S1 with respect to the vertical cross section V, and the upstream pulley 34d on the upstream side of the transmission pulley 20D and on the first side S1 with respect to the vertical cross section V.

As a result, when the support 14 is bent in either direction about the first rotating shaft 16, one or more of the wires 26, 28, 30 and 32 come in contact with the associated one or more of the transmission pulleys 20A to 20D, which stabilizes the controllability when the support 14 is turned. More specifically, when the support 14 is bent from the state illustrated in FIG. 16 toward the second side S2, the wires 26 and 28 come in contact with the transmission pulleys 20A and 20B, respectively. In some embodiments, when the support 14 is bent from the state illustrated in FIG. 17 toward the first side S1, the wires 30 and 32 come in contact with the transmission pulleys 20C and 20D, respectively. Thus, in either case, such a state in which none of the wires are in contact with the transmission pulleys 20A to 20D (run over the transmission pulleys 20A to 20D) is avoided. As a result, the controllability when the support 14 is bent about the first rotating shaft 16 is stabilized.

### Sealing of Base Member

Because the grasping portions 12a and 12b of the forceps device 10 according to some embodiments are operated inside the abdominal cavity of a patient, it is necessary to devise a way to prevent gas around the grasping portions 12a and 12b from leaking out through the forceps device 10 so that the air pressure in the abdominal cavity, which is increased to be higher than the atmospheric pressure, does not lower. In the present embodiment, sealing is therefore provided in a region including the base member 18.

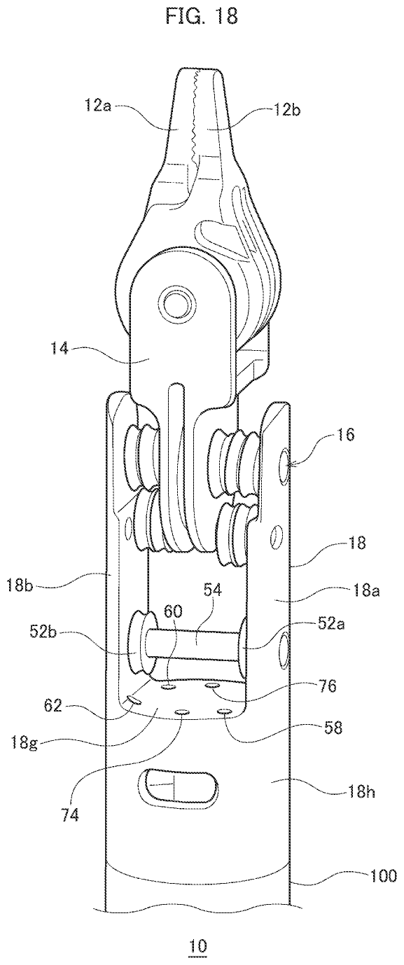

FIG. 18 is a view of the forceps device illustrated in FIG. 1 without illustration of wires. As illustrated in FIG. 18, the forceps device 10 includes the pair of grasping portions 12a and 12b, the support 14 that holds the pair of grasping portions 12a and 12b, the first rotating shaft 16 that turnably supports the support 14, the base member 18 that holds the first rotating shaft 16, a plurality of wires 26, 28, 30 and 32 (not illustrated in FIG. 18; see FIG. 2) for transmitting driving forces to the pair of grasping portions 12a and 12b to move the grasping portions 12a and 12b, a guide pulley 52a for guiding the wire 26, a guide pulley 52b for guiding the wire 32, and a support shaft 54 that rotatably supports the guide pulleys 52a and 52b. The base member 18 is located between the grasping portions 12a and 12b and a shaft 100 of the forceps device 10.

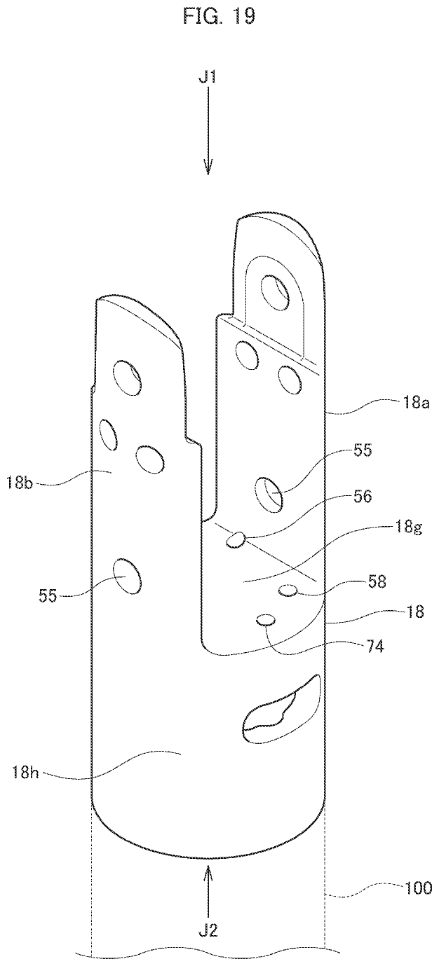

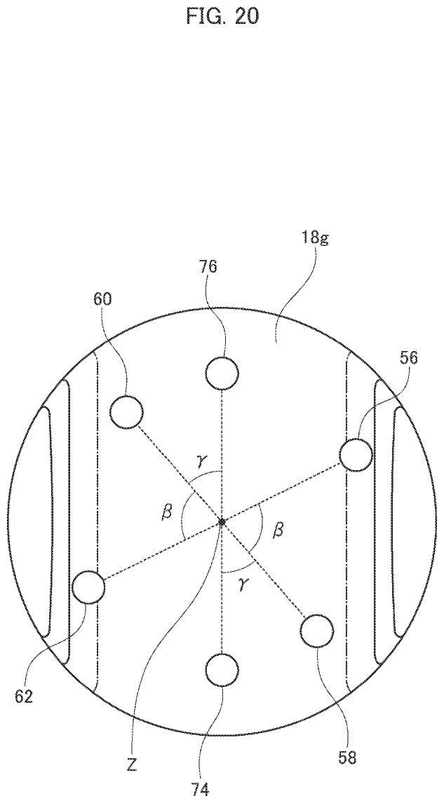

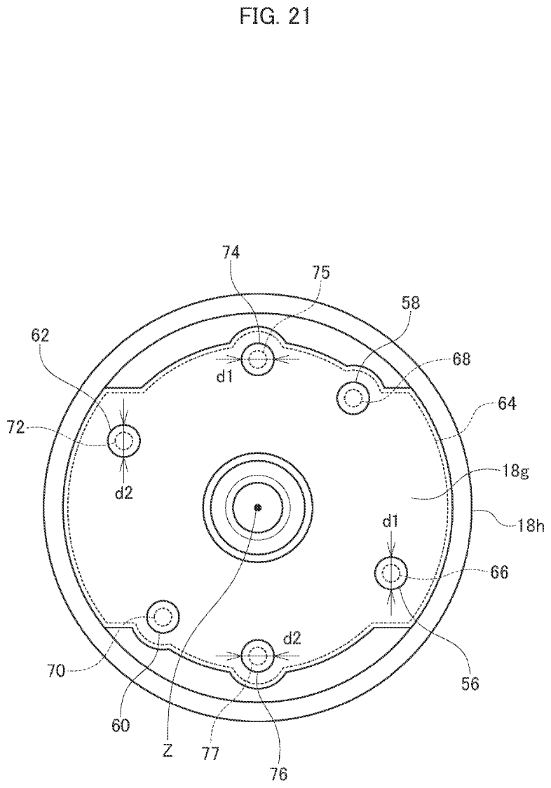

FIG. 19 is a perspective view of the base member according to some embodiments. FIG. 20 is a top view of the base member illustrated in FIG. 19 as viewed in a direction J1. FIG. 21 is a bottom view of the base member illustrated in FIG. 19 as viewed in a direction J2.

The base member 18 includes a partition part 18g having a plurality of grasping-portion-wire holes 56, 58, 60 and 62 through which the wires 26, 28, 30 and 32, respectively, pass, and the pair of arms 18a and 18b which extend from an outer edge of the partition part 18g toward the grasping part (upward in FIG. 19) and to which respective ends of the support shaft 54 are fixed. The arms 18a and 18b each have a fitting hole 55 into which an end of the support shaft 54 that rotatably supports the pair of guide pulleys 52a and 52b is fitted. In this manner, the pair of arms 18a and 18b are fixed via the support shaft 54, which increases the stiffness of the base member 18.

The partition part 18g is a partition formed between a cylindrical part 18h and the pair of arms 18a and 18b. The grasping-portion-wire holes 56 and 58 are a pair of grasping-portion-wire holes that are adjacent to each other at the closet distance, and the grasping-portion-wire holes 60 and 62 are a pair of grasping-portion-wire holes that are adjacent to each other at the closest distance.

As illustrated in FIG. 12, the wires 26 and 28 in a state being guided by the upstream pulleys 34a and 34b are arranged together. Thus, for passing the wires 26 and 28 in this state through the partition part 18g, a large hole through which two wires can pass needs to be formed through the partition part 18g or two small holes through each of which one wire can pass need to be formed through the partition part 18g. In the case of a large hole, the airtightness of sealing with sealing resin, which will be described later, may be lowered. In contrast, in the case of two adjacent small holes, the wall between the holes becomes thin, and the strength of an area of the base member 18 where the holes are formed may therefore be lowered.

Hence, in the forceps device 10 according to some embodiments, the wire 26 is guided by the guide pulley 52a, so that the wire 26 is separated from the wire 28. Similarly, the wire 32 is guided by the guide pulley 52b, so that the wire 32 is separated from the wire 30. In this manner, the grasping-portion-wire hole 56 through which the wire 26 passes and the grasping-portion-wire hole 58 through which the wire 28 passes can be separated from each other. Similarly, the grasping-portion-wire hole 60 through which the wire 30 passes and the grasping-portion-wire hole 62 through which the wire 32 passes can be separated from each other.

In some embodiments, as illustrated in FIG. 21, the base member 18 includes an elastic sealing member 64 (having a type A durometer hardness of 30 to 70) arranged on one side (on one side opposite the side in which the grasping portions 12a and 12b are located) with respect to the partition part 18g. The sealing member 64 is a silicone resin film having a thickness of about 0.5 to 2 mm, for example.

The sealing member 64 illustrated in FIG. 21 has a plurality of sealing holes 66, 68, 70, and 72 at positions corresponding to those of the grasping-portion-wire holes 56, 58, 60 and 62. This enables sealing with the sealing member 64, with the wires 26, 28, 30 and 32 passing through the partition part 18g of the base member 18. The wires have a diameter φ of 0.4 to 0.5 mm, and the diameter d1 of the grasping-portion-wire holes is preferably slightly larger than the wire diameter.

The sealing holes have a diameter d2 that is smaller than the diameter d1 of the grasping-portion-wire holes and smaller than the diameter (wire diameter φ) of the grasping-portion wires. As a result, even when a gap is present between a grasping-portion-wire hole and a grasping-portion wire, the sealing hole and the grasping-portion wire are in close contact with each other, which reduces leakage of gas from the grasping part side to the outside of the forceps device 10 via the base member 18.

As illustrated in the drawings, the four grasping-portion-wire holes according to some embodiments are formed through the partition part 18g at certain distances from each other. Thus, the sealing holes formed at positions corresponding to those of the grasping-portion-wire holes are also separated from the adjacent sealing holes.

In some embodiments, the forceps device 10 according to some embodiments includes the pair of wires 38 and 40 for rotating the support 14 about the first rotating shaft 16 in addition to the wires 26, 28, 30 and 32, which are grasping-portion wires. The partition part 18g of the base member 18 therefore has support-wire holes 74 and 76 through which the wires 38 and 40, respectively, pass. The support-wire holes 74 and 76 are formed at point-symmetric positions with respect to the center Z of the partition part 18g (the central axis of the cylindrical part 18h).

Furthermore, the sealing member 64 has a plurality of sealing holes 75 and 77 formed at positions corresponding to those of the support-wire holes 74 and 76. This enables sealing with the sealing member 64, with the wires 38 and 40 passing through the partition part 18g of the base member 18. The diameter d1 of the support-wire holes is preferably slightly larger than the wire diameter.

### Method for Producing Sealing Member

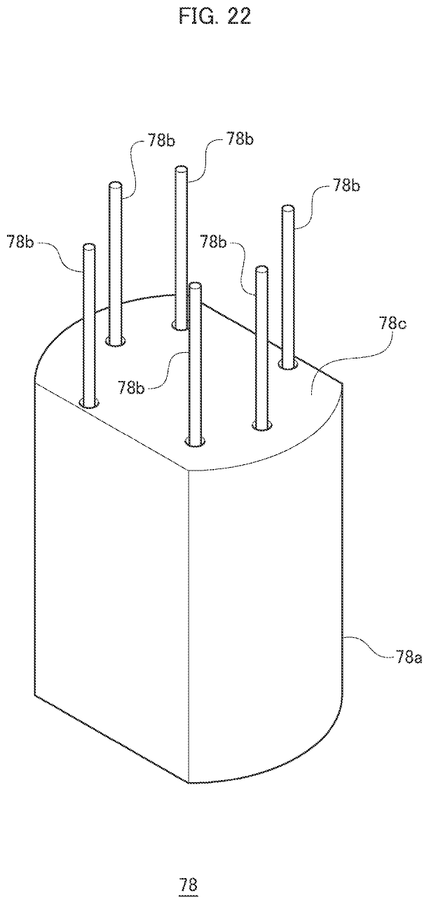

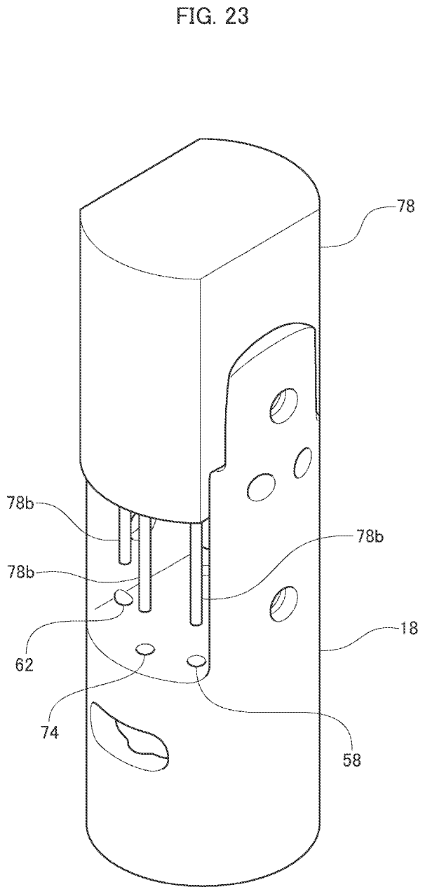

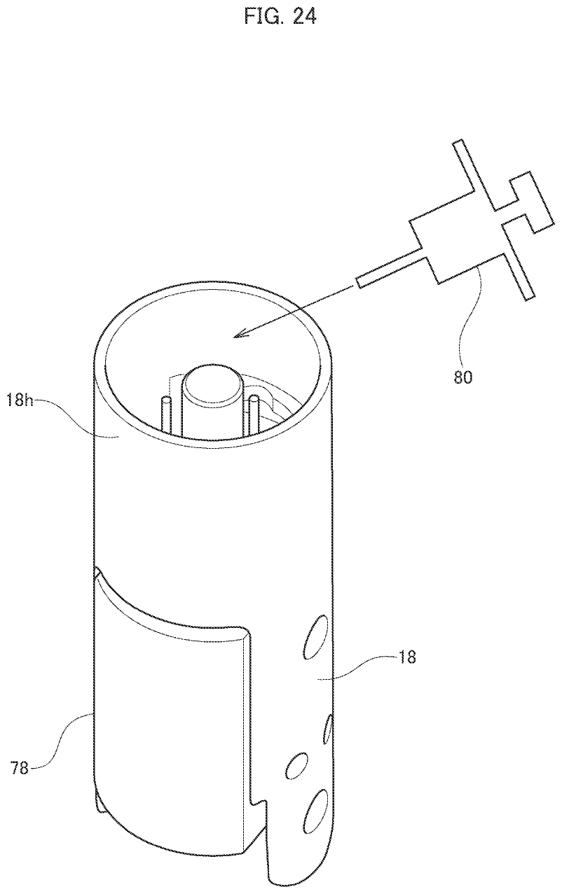

Next, a method for producing the sealing member 64 will be explained. FIG. 22 is a perspective view of a jig to be used for producing a sealing member. FIG. 23 is a perspective view illustrating a state in which the jig illustrated in FIG. 22 is attached to the base member. FIG. 24 is a schematic diagram illustrating injection of sealing resin in a state in which the jig is attached to the base member.

The jig 78 illustrated in FIG. 22 includes a columnar body 78a inserted between the pair of arms 18a and 18b of the base member 18, and six shafts 78b extending from one end face 78c of the body 78a. The six shafts 78b are arranged at predetermined positions on the one end face 78c of the body 78a so that the shafts 78b are inserted into the four grasping-portion-wire holes 56, 58, 60 and 62 and the two support-wire holes 74 and 76 of the partition part 18g.

As illustrated in FIG. 23, the jig 78 is attached between the arms 18a and 18b of the base member 18, and an injection device 80 such as an injector is used to inject sealing resin through an opening of the cylindrical part 18h in a state in which the shafts 78b stick out from a rear face of the partition part 18g. After the sealing resin has hardened and the sealing member 64 is thus molded, the jig 78 is removed, and the sealing holes 66, 68, 70, 72, 75 and 77 are thus formed.

Because the partition part 18g according to some embodiments has six holes formed at dispersed positions, the six shafts 78b of the jig 78 are also arranged at dispersed positions. Thus, the spaces between adjacent shafts 78b are large, and sealing resin injected by the injection device 80 smoothly enters regions between the adjacent shafts 78b. In contrast, if the spaces between adjacent shafts 78b are narrow, there may be cases where viscous sealing resin that has been injected does not smoothly enter regions between adjacent shafts 78b. In such a case, adjacent sealing holes may be integrated, or a sealing hole may not have a predetermined shape.

In the base member 18 according to some embodiments, the holes through the partition part 18g are therefore formed at dispersed positions away from each other so that the sealing holes in the molded sealing member 64 are not close to each other. More specifically, as illustrated in FIG. 20, when the partition part 18g is viewed in the vertical direction, an angle β at the center Z of the partition part 18g between the grasping-portion-wire hole 56 and the grasping-portion-wire hole 58 is equal to or larger than 30° or, more preferably, equal to or larger than 45°, for example.

As a result, the distance between the grasping-portion-wire hole 56 and the grasping-portion-wire hole 58 becomes large, which reduces such defects as integration of holes when the sealing member 64 with a plurality of sealing holes is molded. In other words, the sealing member 64 having the sealing holes is molded with high accuracy on the rear face side of the partition part 18g having the grasping-portion-wire holes and the support-wire holes, and the airtightness on the grasping part side is increased.

Furthermore, when the partition part 18g is viewed in the vertical direction, an angle γ at the center Z of the partition part 18g between the support-wire hole 74 and the grasping-portion-wire hole 58 that is closest to the support-wire hole 74 is equal to or larger than 30° or, more preferably, equal to or larger than 35°, for example. As a result, the support-wire hole 74 and the grasping-portion-wire hole 58 can be separated from each other. Thus, the sealing member 64 having the sealing holes is molded with high accuracy on the rear face side of the partition part 18g having the grasping-portion-wire holes and the support-wire holes, and the airtightness on the grasping part side is increased.

While various embodiments have been described above with reference to the drawings, the present disclosure is not limited thereto, and any combination or substitution of components as appropriate is included within the scope of the present disclosure. In some embodiments, modifications such as combinations, changes in the order of processes, and various changes in design may be made on the basis of knowledge of a person skilled in the art, and such modified embodiments are within the scope of the present disclosure and the appended claims.

Embodiments consistent with the present disclosure can be used for a manipulator of a surgical robot.

1. A forceps device comprising:
a grasping part;
a support that holds the grasping part;
a rotating shaft that turnably supports the support;
a base member that holds the rotating shaft;
a plurality of grasping-portion wires that transmit driving forces to move the grasping part;
a guide pulley that guides one or more of the plurality of grasping-portion wires; and
a support shaft that rotatably supports the guide pulley,
wherein the base member includes:
a partition part having a plurality of grasping-portion-wire holes through which the respective grasping-portion wires pass; and
a pair of arms extending from the partition part toward the grasping part, respective ends of the support shaft being fixed to the respective arms,
wherein the plurality of grasping-portion wires include a first grasping-portion wire and a second grasping-portion wire,
wherein the plurality of grasping-portion-wire holes include a first grasping-portion-wire hole through which the first grasping-portion wire passes, and a second grasping-portion-wire hole which is closest to the first grasping-portion-wire hole among the plurality of grasping-portion-wire holes and through which the second grasping-portion wire passes, and
wherein the guide pulley guides the one or more of the plurality of grasping-portion wires so that the first grasping-portion wire is separated from the second grasping-portion wire.

2. The forceps device according to claim 1, wherein:
when the partition part is viewed in a vertical direction, an angle at a center of the partition part between the first grasping-portion-wire hole and the second grasping-portion-wire hole is equal to or larger than 30°.

3. The forceps device according to claim 2, wherein:
the base member includes a sealing member on one side with respect to the partition part, and
the sealing member has a plurality of sealing holes at positions corresponding to the plurality of grasping-portion-wire holes.

4. The forceps device according to claim 3, wherein:
the plurality of sealing holes each have a diameter that is smaller than a diameter of a corresponding one of the plurality of grasping-portion-wire holes and smaller than a diameter of a corresponding one of the plurality of grasping-portion wires.

5. The forceps device according to claim 4, wherein:
the sealing member is a silicone resin film.

6. The forceps device according to claim 3, wherein:
the sealing member is a silicone resin film.

7. The forceps device according to claim 1, wherein:
the base member includes a sealing member on one side with respect to the partition part, and
the sealing member has a plurality of sealing holes at positions corresponding to the plurality of grasping-portion-wire holes.

8. The forceps device according to claim 1, further comprising a pair of support wires that transmit driving forces to turn the support,
wherein
the partition part has a pair of support-wire holes through which the pair of support wires, respectively, pass, and
when the partition part is viewed in a vertical direction, with respect to a center of the partition part, an angle between each of the pair of support-wire holes and a grasping-portion-wire hole among the plurality of grasping-portion-wire holes that is closest to the support-wire hole is equal to or larger than 30°.

9. Abase member arranged between a grasping part of a forceps device and a shaft of the forceps device, the base member comprising:
a partition part having a plurality of grasping-portion-wire holes through which a plurality of grasping-portion wires, respectively, pass, the plurality of grasping-portion wires transmitting driving forces to move the grasping part,
wherein:
the plurality of grasping-portion-wire holes include a first grasping-portion-wire hole and a second grasping-portion-wire hole which is closest to the first grasping-portion-wire hole among the plurality of grasping-portion-wire holes, and
when the partition part is viewed in a vertical direction, with respect to a center of the partition part, an angle between the first grasping-portion-wire hole and the second grasping-portion-wire hole is equal to or larger than 30°.

10. The base member according to claim 9, wherein:
the partition part has a pair of support-wire holes through which a pair of support wires, respectively, pass, the pair of support wires transmitting driving forces for turning a support of the forceps device that holds the grasping part, and
when the partition part is viewed in the vertical direction, with respect to the center of the partition part, an angle between each of the pair of support-wire holes and a grasping-portion-wire hole among the plurality of grasping-portion-wire holes that is closest to the support-wire hole is equal to or larger than 30°.

11. The base member according to claim 10, further comprising a pair of arms extending from the partition part toward the grasping part,
wherein the pair of arms each have a fitting hole, and
respective ends of a support shaft of the forceps device that rotatably supports a pair of guide pulleys of the forceps device being fitted into the fitting holes.

12. The base member according to claim 9, further comprising a pair of arms extending from the partition part toward the grasping part,
wherein the pair of arms each have a fitting hole, and
respective ends of a support shaft of the forceps device that rotatably supports a pair of guide pulleys of the forceps device being fitted into the fitting holes.

13. A forceps device comprising:
a first grasping portion and a second grasping portion;
a support that holds the first grasping portion and the second grasping portion;
a first shaft that turnably supports the support;
a base member that holds the first shaft;
a first wire and a second wire that transmit driving forces to move the first grasping portion and the second grasping portion;
a first guide pulley and a second guide pulley; and
a support shaft that rotatably supports the first guide pulley and the second guide pulley,
wherein the base member includes:
a partition part having a plurality of holes including a first hole through which the first wire passes and a second hole that is closest to the first hole among the plurality of holes and through which the second wire passes; and
a first arm and a second arm extending from the partition part toward the first grasping portion and the second grasping portion, respective ends of the support shaft being fixed to the first arm and the second arm, and the first guide pulley being provided adjacent to the first arm and the second guide pulley being provided adjacent to the second arm and spaced apart from the first guide pulley.

14. The forceps device according to claim 13, wherein:
when the partition part is viewed in a vertical direction, with respect to a center of the partition part, an angle between the first hole and the second hole is equal to or larger than 30°.

15. The forceps device according to claim 14, wherein:
the base member includes a sealing member, and
the sealing member has a plurality of sealing holes at positions corresponding to the plurality of holes in the partition part.

16. The forceps device according to claim 15, wherein:
the plurality of sealing holes each have a diameter that is smaller than a diameter of a corresponding one of the plurality of holes in the partition part and smaller than a diameter of each of the first wire and the second wire.

17. The forceps device according to claim 16, wherein the sealing member is a silicone resin film.

18. The forceps device according to claim 15, wherein the sealing member is a silicone resin film.

19. The forceps device according to claim 13, wherein:
the base member includes a sealing member, and
the sealing member has a plurality of sealing holes at positions corresponding to the plurality of holes in the partition part.

20. The forceps device according to claim 13, further comprising a first support wire and a second support wire that transmit driving forces to turn the support,
wherein:
the partition part includes a first support hole and a second support hole through which the first support wire and the second support wire pass, respectively, and
when the partition part is viewed in a vertical direction, with respect to a center of the partition part, an angle between the first support hole and a closest one of the plurality of holes to the first support hole is equal to or larger than 30°, and an angle between the second support hole and a closest one of the plurality of holes to the second support hole is equal to or larger than 30°.
